(19) United States
(12) Patent Application Publication
(10) Pub. No.: US 2008/0221556 A1
(43) Pub. Date: Sep. 11, 2008

(54) Device For the Controlled Release of a Predefined Quantity of a Substance

(75) Inventors: Mark Thomas Johnson, Eindhoven (NL); Ralph Kurt, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., Eindhoven (NL)

(21) Appl. No.: 11/994,443

(22) PCT Filed: Jun. 30, 2006
(86) PCT No.: PCT/IB06/52202

§ 371 Date: Jan. 2, 2008

(30) Foreign Application Priority Data
Jul. 5, 2005 (EP) 05106082.0

(51) Int. Cl. A61K 9/22

(52) U.S. Cl. 604/890.1

(57) ABSTRACT

The application provides a device (10) for the controlled release of a predefined quantity of a substance and a method for controllably releasing a predefined quantity of a substance from a compartment. The device comprises a matrix arrangement of compartments (20) in a substrate, each compartment being closed by at least one release mechanism, at least one first electrode (40) and at least one second electrode (50) being assigned to each compartment, the device comprising a plurality of row selection lines (60) and a plurality of column selection lines (70) and a plurality of column selection lines, the number of compartments exceeding the sum of the number of row selection lines and the number of column selection lines, each first electrode being electrically connected directly to one of the plurality of row selection lines and each second electrode being electrically connected directly to one of the plurality of column selection lines.

The present invention relates a device for the controlled release of a predefined quantity of a substance. The present invention further relates to a method for controllably releasing a predefined quantity of a substance from a compartment.

Accurate delivery of small, precise quantities of one or more chemicals into a carrier fluid are of great importance in many different fields of science and industry. Examples in medicine include the delivery of drugs to patients using intravenous methods, by pulmonary or inhalation methods or by the release of drugs from vascular stent devices. Examples in diagnostics include releasing reactions into fluids to conduct DNA or genetic analysis, combinatorial chemistry, or the detection of a specific molecule in an environmental sample. Other applications involving the delivery of chemicals into a carrier fluid include the release of fragrances and therapeutic aromas from devices into air and the release of flavoring agents into a liquid to produce beverage products.

Devices for the controlled release of a predefined quantity of a substance are generally known. For example, the US patent application US 2004/0034332 A1 discloses an implantable device for controlled delivery of a drug, the device including a microchip which have reservoirs containing the molecules for release. The microchip device includes a substrate, at least two reservoirs in the substrate containing the molecules for release and a reservoir cap positioned on or within a portion of the reservoir and over the molecules, so that the molecules are controllably released from the device by diffusion through or upon disintegration or rupture of the reservoir caps. Each of the reservoirs of a single microchip can contain different molecules which can be released independently. One drawback of the known device is that each reservoir is directly contacted to an electrode which is used to electrically break the seal layer or the cap by applying a current and to release the drug. A weakness of the prior art system is that one external electrical connection is required for each compartment or for each reservoir from which the drug is to be released. This strongly limits the number of compartments, which can be realized on a single device as the space required for all the electrical connections becomes prohibitive.

It is therefore an object of the present invention to provide a device for the controlled release of a predefined quantity of a substance that has an increased number of reservoirs or compartments without the need for providing one external electrical connection for each compartment to be controlled independently.

The above object is achieved by a device and a method for the controlled release of a predefined quantity of a substance according to the present invention. The device for the controlled release of a predefined quantity of a substance comprises a matrix arrangement of compartments in a substrate, each compartment being closed by at least one release mechanism, at least one first electrode and at least one second electrode being attributed to each compartment, the device comprising a plurality of row selection lines and a plurality of column selection lines, the number of compartments exceeding the sum of the number of row selection lines and the number of column selection lines, each first electrode being electrically connected directly to one of the plurality of row selection lines and each second electrode being electrically connected directly to one of the plurality of column selection lines, wherein the release mechanism has a threshold behavior.

An advantage of the apparatus according to the invention is that it is possible to realize a controlled substance or drug delivery system based upon a multiplicity of individual drug release compartments where the number of compartments is very high, i.e. in the range of 1.00-1,000,000 compartments. According to the prior art, the number of compartments is strongly limited by the need to contact each compartment individually by a connecting line.

A further advantage of the present invention is that the control of delivery of a substance or a drug is based upon a passive matrix principle. This is in contrast to the prior art systems where each compartment is directly connected to an electrical connection. By the use of a passive matrix, it is feasible to release drugs from any of the large number of compartments of the order of 1.00-1,000,000 in a controlled manner. This is not feasible if every compartment were to be individually controlled by a dedicated control device as the costs and space required to incorporate such a control system would be prohibitive. A further advantage of the present invention is that thereby, applications such as for example external drug delivery systems (patches), implantable drug delivery systems or oral drug delivery systems (e-pill) are possible. A drug delivery system according to the present invention may be applied for delivery of a single drug, but can be advantageously applied to a system where several different drugs are applied from the same array or the same device. A passive matrix type device for the controlled release of a predefined quantity of a substance is realized by electrically connecting directly each compartment or at least each release mechanism of a compartment or at least two electrodes associated or attributed to a compartment to one of a plurality of row selection lines and to one of a plurality of column selection lines. The passive matrix principle is realized by connecting directly the electrodes (first and second electrode attributed to each compartment) to the row selection lines or the column selection lines without any active electrical component.

According to the invention, a drug release or cap removing mechanism is required, which shows a threshold behavior or at least a strongly non-linear behavior with respect to the release signal coming from the row selection lines and/or the column selection lines. In the context of the present invention, a threshold behavior is at least a strongly non-linear behavior of the sealing capacity of the release mechanism, e.g. a closure cap, with respect to the release signal. If a release signal (e.g. 0.5 V in the case of an electrical potential difference used as a release signal) is applied below a threshold value of the release signal (e.g. 0.75 V in the case of an electrical potential difference used as a release signal), then the resulting effect on the sealing capacity (or the loss of sealing capacity of the release mechanism) is negligible. Only if a release signal (e.g. 1.0 V in the case of an electrical potential difference used as a release signal) above such a threshold value of the release signal is applied, is the sealing capacity of the release mechanism strongly modified, i.e. reduced.

In a preferred embodiment of the present invention, the release mechanism is a one-time release mechanism. This means that the release mechanism is in some manner “destroyed” by applying a release signal above the threshold and the release mechanism is not re-usable. Thereby, it is possible to provide the release mechanism very cost-effectively and easy to manufacture. Nevertheless, the present invention also refers to a release mechanism which is closable once it has been opened (for the first time) and further on re-openable at least a second time. Such an embodiment employing a re-closable and re-openable release mechanism is less preferred because it usually implies higher costs.

Very preferably, the release mechanism according to the present invention is provided by means of a closure cap. A closure cap is one specific and preferred embodiment of realizing a release mechanism. Examples of other release mechanisms are: a polymer membrane or a gel that releases drugs if heated (decomposition of a carrier matrix or changing properties of it, such as breaking dedicated chemical bonds) or membranes that change their permeability for certain molecules upon applying an electrical potential.

In a preferred embodiment of the present invention, each compartment is defined by means of one specific row selection line out of the plurality of row selection lines and one specific column selection line out of the plurality of column selection lines. As a result, the matrix principle for addressing an individual compartment is realized and therefore the number of connection lines strongly reduced.

In a further preferred embodiment, the number of compartments is in the order of magnitude of the number of row selection lines multiplied by the number of column selection lines. It is therefore possible to reduce the required connecting line on the device even more and therefore render the device smaller, of lighter weight and more cost-effective.

In a further preferred embodiment, the number of row selection lines is substantially the same as the number of column selection lines. It is therefore possible to further reduce the required connecting lines on a device with a given number of capsules and therefore render the device even smaller, of lighter weight and even more cost-effective.

In a still further preferred embodiment of the present invention, a first group of compartments is provided to contain a first quantity of a first substance and a second group of compartments is provided to contain a second quantity of a second substance. An advantage of the device according to the present invention is that a very flexible substance release mechanism can be implemented in the structure of the inventive device. For example, it is possible to provide compartments of different size, thereby being able to contain different volumes of the substance or substances to release. For example, if at a given moment a greater quantity of a substance is to be released, a device can be controlled accordingly and open a compartment having an appropriate size and hence an appropriate volume of the substance to be released. This is instead of releasing the same quantity of substance from a certain number of smaller compartments which would have the same effect. Of course, the release of an appropriate quantity of a substance out of one single compartment is easier to control and therefore makes the device according to the present invention smaller, of lighter weight and more cost-effective. Accordingly, the first and second substance can be different or identical. Another way to improve the flexibility of releasing substances like drugs or the like is to provide several different substances or different mixtures of substances in different compartments on the device, the different compartments being of the same or a different size. It is thereby possible to controllably release for example two different drugs alternatively during the day or during another time interval to the patient. Alternatively, it is also possible to further enhance the flexibility of use of the inventive device for example by providing differently sized compartments as well as different substances in the differently sized compartments. It is preferred according the present invention, that the first quantity is approximately half of the second quantity. It is thereby also possible to have a first group of compartments having a first volume or containing a first quantity of a substance, a second group of compartments containing each twice the first quantity, a third group containing four times the first quantity and a fourth group of compartments containing eight times of the first quantity. Thereby flexibility of releasing one or more substances is even further enhanced.

In a preferred embodiment of the present invention, the release mechanism of the compartment is provided removable or disintegratable by applying an electrical potential between the first electrode and the second electrode. It is then possible to very easily and quickly control the release of the substance out of one of the compartments.

In a further preferred embodiment the first electrode and the second electrode of each compartment are provided substantially electrically insulated from each other. Very advantageously it is thereby possible that interference effects are reduced by selecting a row and a column to define a specific compartment on the matrix arrangement of compartments on the device. The release of a substance out of the compartments can then be controlled with the inventive device very precisely.

It is further preferred, that the release mechanism is activated by means of an electro-chemical reaction or by means of heating the release mechanism, preferably by means of an electrical current. The device can be produced in a very cost-effective manner and the release of the substance can be made quicker and more accurate.

Further embodiments of the present invention are provided with a control unit for controlling the release of the substance. It is further preferred, that the number of compartments is at least 100, preferably at least 1,000, more preferably at least 10,000, still preferably at least 100,000 and most preferably at least 1,000,000 compartments.

The compartments could be filled by micropipette or ink jet printing techniques.

The present invention also includes a method for controllably releasing a predefined quantity of a substance from a compartment using a device comprising a matrix arrangement of compartments in a substrate, each compartment being closed by at least one release mechanism, at least one first electrode and at least one second electrode being assigned to each compartment, the device comprising a plurality of row selection lines and a plurality of column selection lines, the number of compartments exceeding the sum of the number of row selection lines and the number of column selection lines, the method comprising the steps of:

electrically connecting the first electrode of the compartment directly to one of the plurality of row selection lines,

electrically connecting the second electrodes of the compartment directly to one of the plurality of column selection lines,

applying an electrical potential above a predetermined threshold value between the first electrode and the second electrode by means of one of a plurality of row selection lines and one of a plurality of column selection lines.

It is thereby possible to controllably release a specific quantity of a substance in a very rapid and easily controlled manner.

In a preferred embodiment of the method according to the present invention, more than one compartment release the substance at the same time. This may mean that more than one compartment are opened simultaneously and that the period of releasing the substance or the drug is then common for each of these compartments. Alternatively, it is also possible that a plurality of compartments are opened sequentially such that their period of release (usually much longer than the time required for opening a specific compartment) overlap and a release of the substance by more than one compartment is possible. It is thereby possible to very flexibly control the release of a substance.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

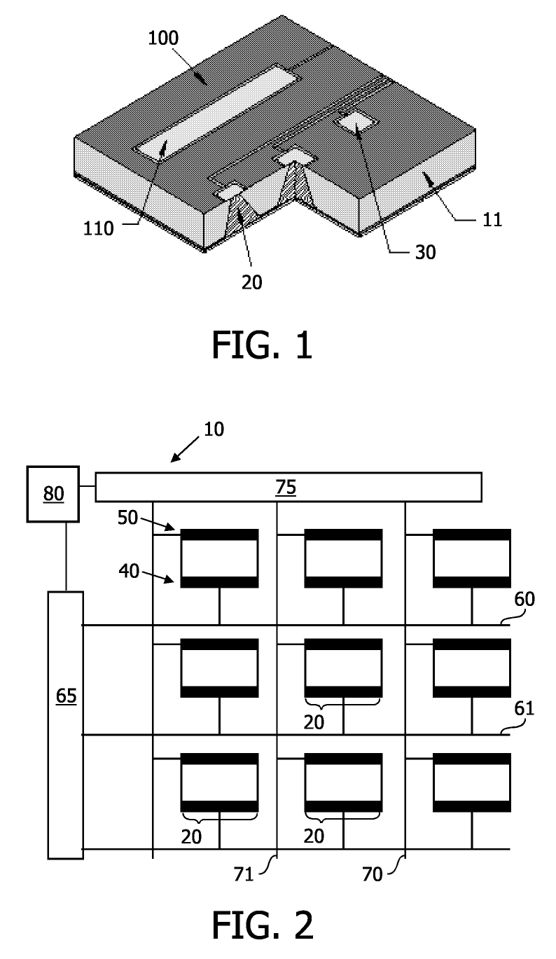

FIG. 1 illustrates schematically a device 100 according to the prior art showing a basic structure of a device of such a type.

FIG. 2 illustrates schematically a device according to the present invention.

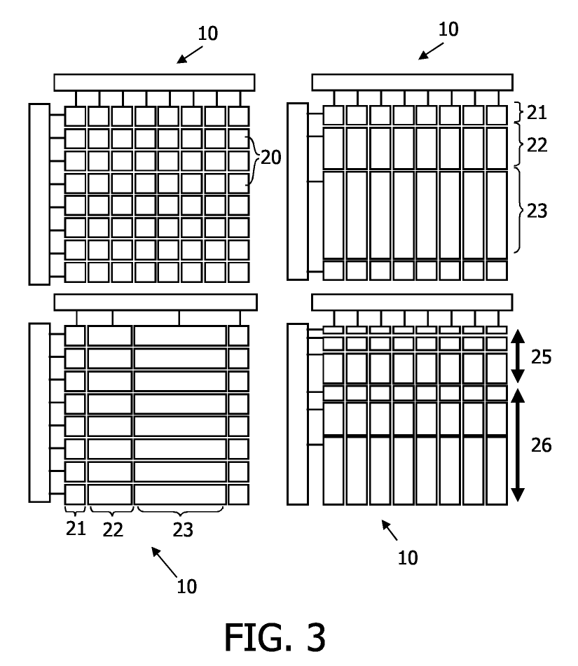

FIG. 3 illustrates four different arrangements of compartments in a device according to the present invention.

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn to scale for illustrative purposes.

Where an indefinite or definite article is used before a singular noun, e.g. “a”, “an”, “the”, this includes a plural of that noun, unless otherwise specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting of only components A and B. It means that with respect to the present invention, the only relevant components of the device are A and B.

In FIG. 1, a known device 100 according to the prior art is schematically shown. The known device 100 comprises a substrate 11 where a plurality of compartments 20 are located. The compartments 20 are closed by a release mechanism 30, especially a closure cap 30. It can further be seen from FIG. 1 that there are electrode lines running to each of the compartments 20 or at least to or near to each of the release mechanisms 30. The connecting lines are not described with a reference sign in FIG. 1. The known device 100 further comprises an electrode area 110.

In FIG. 2 an inventive device 10 is schematically shown comprising a plurality of compartments 20 where only nine compartments 20 are shown. The device 10 comprises the compartments 20 in a substrate 11 comparable to the prior art devices. The substrate 11 is the structural body in which the compartments 20 are formed, e.g. it contains the etched, machined or molded compartments 20. A compartment 20 (which is also called a reservoir in the following) is a container for a substance. Micro-electromechanical system methods, micro-molding and micro-machining techniques known in the art can be used to fabricate the substrate 11 together with the compartments 20 from a variety of materials. Examples of suitable substrate materials include metals, ceramics, semiconductors, degradable and non-degradable polymers. Bio-compatibility of the substrate material typically is preferred for in-vitro device applications. The substrate, or portions thereof, may be coated, encapsulated, or otherwise contained in a bio-compatible material before use. The substrate 11 can be flexible or rigid. In one embodiment, the substrate 11 serves as a support for a microchip device. In one example, the substrate 11 is formed of silicon. The substrate 11 can have a variety of shapes for shaped surfaces. It can, for example, have a release side, i.e. an area having release mechanisms, that is planar or curved. The substrate may for example be in a shape selected from discs, cylinders, or spheres. In one embodiment, the release side can be shaped to conform to a curved tissue surface. This would be particularly advantageous for local delivery of a therapeutic agent to that tissue surface. In another embodiment the backside (distal to the release side) is shaped to conform to an attachment surface. The substrate may consist of only one material or may be a composite or multi-laminate material, that is, composed of several layers of the same or different substrate materials that are bonded together.

In the schematical illustration of FIG. 2 of the inventive device 10, the inventive device 10 comprises for each compartment 20 a first electrode 40 and a second electrode 50. Preferably, the first and the second electrodes 40, 50 are not directly electrically connected, i.e. they are substantially insulated from each other by e.g. a dielectric medium such as a fluid. This means, that the electrical resistance created by materials separating the first and second electrode 40, 50 from each other are from a sufficiently high resistivity that regarding the applied voltages or potential differences there is no substantial current flow between the first and second electrode 40, 50. The inventive device 10 further comprises the compartments 20 in the form of a matrix arrangement. Further, the inventive device 10 comprises a plurality of row selection lines 60 and a number of column selection lines 70. Here, the row and column selection lines are shown in a mutually perpendicular alignment, while other matrix arrangements, such as on a hexagonal or triangular grid would also be possible, providing the row and column lines are configured in mutually different orientations. For the sake of example, one specific row selection line 61 from the plurality of row selection lines 60 is specifically shown in FIG. 2. Accordingly, one specific column selection line 71 from the plurality of column selection lines 70 is shown in FIG. 2. The specific row selection line 61 and column selection line 71 of FIG. 2 define the compartment 20 in the middle of the matrix arrangement of nine compartments 20 shown in FIG. 2. This means, that by selecting the specific row and column selection lines 61, 71, the compartment in the middle of the matrix arrangement is selected for being activated. This means, that the release mechanism 30 would be removed or activated by applying an appropriate electrical signal to the specific row and column selection lines 61, 71. For the sake of clarity the release mechanism 30 is not depicted in FIG. 2, but would be associated with one of the electrodes. In contrast a first driver 65 for driving the row selection lines 60 is shown in FIG. 2 as well as a second driver 75 for driving the column selection lines 70. Furthermore, a control unit 80 for controlling the release of the substance is also shown in FIG. 2. The control unit 80 controls the first and second driver 65, 75 for defining a specific compartment 20 by means of specific row selection lines 61, 71. The control unit 80 also controls the successive activation of different compartments 20. This means, that the control unit 80 for example controls the opening of the release mechanisms 30 of different compartments such that for example the concentration of a drug remains at an optimum therapeutic level during the course of a treatment. As the optimum concentration of the drug is variable from one patient to the next, and during the course of the treatment, it is necessary that this drug delivery system is extremely flexible and provides an almost continuously variable dosage of the drug. (Such a system could be used e.g. for chronotherapy) Such a drug release system is possible to realize with the inventive device. Preferably the control unit 80 either has sensors for determining the actual level of the drug in the environment of the device 10 or the device 10 is coupled to such a sensor device (not shown) such that a signal from the sensor device signalling the control unit 80 to increase or decrease drug release results in an appropriate reaction of the inventive device, i.e. the control unit 80 activates the first and second drivers 65, 75 in order to increase or decrease the release of the substance inside the compartments 20.

In order for a passive matrix system to operate successfully, it is required that the substance or drug release mechanism shows a threshold behavior, i.e. at least a strongly non-linear behavior with respect to the release signal. As an example, if the drug delivery i.e. the opening of the release mechanisms 30 is based upon an electro-chemical reaction which breaks the seal of the compartment 20 or which breaks the release mechanism 30 of the compartment 20, and where a voltage of a around 1 V is required to initiate the electro-chemical reaction, a voltage of around 0.5 V will be insufficient to initiate the electro chemical reaction. For example, either of the first and second electrodes is provided as a cathode and the other electrode of the first and second electrodes serves as an anode. The anode is defined as the electrode where oxidation occurs. Any conductive material capable of dissolving into solution or forming soluble ions or oxidation compounds upon application of an electric current or an electric potential (electrochemical dissolution) can be used for the fabrication of the anodes and cathodes. In addition, materials that normally form insoluble ions of oxidation products in response to an electric potential can be used if, for example, local pH changes near the anode cause these oxidation products to become soluble. Examples of suitable reservoir cap materials include metals such as copper, gold, silver, and zinc, and some polymers.

In the example of FIG. 2, the selection of one compartment 20 out of the multitude of compartments 20 by means of selecting one specific row selection line 61 and a specific column selection line 71 can be made by applying for example ˜0.5 V to the specific row selection line 61 and to apply the voltage of +0.5 V to the specific column selection line 71. This results in applying a sufficient electrical potential between the first and the second electrode 40, 50 of one specific compartment 20 which is in FIG. 2 located in the middle of the matrix arrangement of compartments 20. The voltage between the first and second electrode 40, 50 of the selected compartment 20 is then amounting to 1 V thus initiating the drug release. The voltage in the other compartments 20 is therefore held at a voltage which will not release the drug. After the drug or the substance is released, the row selection line 60 and the column selection line 70 are again set to 0 V which corresponds also to the rest state of the inventive device 10 thereby saving electrical power. In this example, the row electrodes or row selection lines 60 might be connected to the first driver 65 which can be realized by a standard low-voltage shift register similar to a gate driver for an active matrix liquid crystal display. Such a voltage shift register can for example switch between 0 V and −0.5 V. The second driver 75 could be just a standard voltage data driver as used e.g. for passive or active matrix liquid crystal displays with an output which may have either 0 V or +0.5 V levels.

In one embodiment of the present invention it is also possible to release a drug or a substance from more than one compartment in a given row simultaneously by applying a release signal to more than one row, i.e. more than one specific row selection line 61 in the array. Then different compartments 20 are simultaneously selected as being active, i.e. as being opened through removing the release mechanism 30 or by disintegrating the release mechanism 30. Accordingly it is also possible to simultaneously or sequentially release drugs from compartments 20 in different columns by activating a specific row selection line 61 and applying a release signal to one or more columns in the array.

In another embodiment of the present invention, the drug delivery mechanism, i.e. the mechanism for opening the release mechanism 30, is based upon a heating effect, i.e. the heating of the release mechanism 30 breaks the release mechanism 30 of the compartment 20 which is selected. As the heat generation is a quadratic function of the applied voltage between the first and second electrode 40, 50, a voltage of around half the release voltage will supply only 25% of the required heating to the compartment 20 which will be insufficient to break or to open the release mechanism 30.

When the release mechanism, i.e. the opening mechanism of the closure cap 30 is provided as an electro-chemical reaction, the first or second electrode 40, 50 can for example be provided as a gold layer in the vicinity of the release mechanism 30. The other one of the first and/or second electrode 40, 50 is for example another metallized electrode. By applying a voltage between the first and second electrodes 40, 50 a gold layer or gold cap acts as an anode in an electro-chemical reaction and is dissolved when a sufficiently high voltage is applied. When the gold layer or the gold cap is removed, then either the closure cap 30 is also removed because the closure cap 30 consists essentially of the gold cap, or the removing of the gold cap sufficiently weakens the release mechanism 30 made of another material so that the release mechanism 30 will break if the gold cap is removed. Anyway, after the electrochemical reaction has taken place, the substance or drug inside the compartment 20 is freed and allowed to diffuse away. In such an embodiment of the inventive device, the substrate 11 is for example provided in the form of a silicon wafer containing the compartments 20 as micro reservoirs which are etched into the silicon substrate. Alternative substrate materials include glass, metals and polymers.

According to a feature of any of the described embodiments of the present invention, the substrate 11 or the chip can be packaged with a battery and a micro processor or a control unit to be completely self-contained. Preferably the control unit 80 is monolithically integrated with the substrate 11 having the compartments 20.

The compartment 20 contents comprise essentially any object or material that needs to be isolated (e.g. protected from) the environment outside the compartment 20 until a selected point in time, when its release or exposure is desired. In various embodiments, the compartment 20 contents comprise a certain quantity of molecules or of a specific substance or of a mixture of specific substances. Proper functioning of certain reservoir contents such as a catalyst or a sensor generally does not require the release of the compartment contents. Rather, their intended function, e.g. catalyzes or sensing, occurs upon exposure of the reservoir contents to the environment outside the compartment 20 after opening of the release mechanism 30. Thus, the catalyst molecules or sensing component can be released or can remain immobilised within the open compartment 20. Other compartment contents such as drug molecules may often need to be released from the compartment in order to pass from the device and be delivered to a site in vivo to exert a therapeutic effect on a patient. However, the drug molecules may be retained for certain in vitro applications. The compartment 20 contents can include essentially any natural or synthetic, organic or inorganic molecule or mixture thereof. The molecules may be in essentially any form, such as a pure solid or liquid, a gel or hydrogel, a solution or emulsion, a slurry or a suspension. The molecules of interest may be mixed with other materials to control or enhance the rate and/or time of release of an open compartment 20. In various embodiments, the molecules may be in the form of solid mixtures, including amorphous or crystalline mixed powders, monolithic solid mixtures, lyophilized powders and solid interpenetrating networks. In other embodiments, the molecules are in liquid forms, such as solutions, emulsions, colloidal suspensions, slurries or gel-mixtures such as hydrogels.

In FIG. 3 four different arrangements of compartments 20 within an inventive device 10 are schematically depicted. In a first embodiment of the device 10 (see FIG. 3 top left) all the compartments 20 are of the same size and provided in a matrix arrangement. The size of the compartments 20 defines a first quantity of a substance contained in the compartments 20. It is either possible that all compartments 20 contain the same substance or it is possible that in a first group (not shown) of the compartments 20, a first substance is located and that in a second group (not shown) of the compartments 20 a second substance is located.

In the second example shown in FIG. 3 (see FIG. 3 top right) an inventive device 10 is depicted where a first group 21 of compartments 20 has a predefined size, allowing to contain a first quantity of a substance. A second group 22 of compartments 20 comprises compartments 20 which are larger than the compartments 20 of the first group 21. Thus, the compartments of the second group 22 are for example able to contain a second quantity of a substance which is twice the first quantity. Of course every other ratio of the first and second quantities is also possible. A third group 23 of compartments 20 comprises compartments 20 which are able to contain a third quantity of a substance. The third quantity being for example twice the second quantity and four times the first quantity. Of course the third quantity can also be provided in a different ratio regarding the first and the second quantity. By selecting specific compartments 20 from the first the second or the third group 21, 22, 23 of compartments 20 it is possible according to the present invention to release a higher or lower amount or quantity of a substance out of the compartments 20 by means of just opening one single compartment 20. This has the advantage that the release of different quantities of the substance is possible to control very easily and with small efforts especially regarding the control unit 80.

In a third example of the inventive device 10 of the present invention depicted in FIG. 3 (see FIG. 3 bottom left) a matrix arrangement of compartments 20 with different groups 21, 22, 23 of compartments 20 is shown. In the third example the arrangement of compartments 20 is comparable to the arrangement of compartments 20 in the second example (FIG. 3 top right). In the third example the size of compartments in each row of the matrix arrangement is identical, whereas the different groups of compartments are realized by changing the size of compartments 20 between different columns. In contrast, in the second example (see FIG. 3 top right) the compartments of each column are identically sized and the compartments of different rows are different.

In a fourth example of a matrix arrangement of the compartments 20 in an inventive device 10 according to the present invention, there is defined a first area 25 of compartments 20 which contains a first substance and there is defined a second area 26 of compartments 20 which contains a second substance.

By the examples given of different matrix arrangement of the compartments 20 of an inventive device, it is possible to have a high flexibility in dosing different quantities and/or different substances by means of the inventive device 10. By changing the size of the compartments 20 and hence the quantities of substances released, a more flexible drug delivery is possible with a smaller number of compartments. For example by providing compartments of sizes in the range from 1:2:4:8:16 etc. it is possible to provide a wide range of dosing a simultaneously opening one or more compartments 20 in a controlled manner. In the case of the delivery more than one type of substance (see example four of FIG. 3, bottom right) it is usual that different drugs have different dosing quantities. For this reason it will be preferred to have different sections or areas 25, 26 of the matrix array of compartments 20 with proportionally larger or smaller compartments 20 depending upon the drug to be delivered. This is preferably achieved by uniformly increasing this spacing between row selection line 60 and/or column selection line 70 in the array as is illustrated in FIG. 3 (bottom right) as this makes the best use of the available drivers 65, 75 and to reduce redundancies of elements included in the device 10. Depending on the complexity of the desired device 10, a memory or shift register is needed to keep the status of the compartments 20 used and still available updated. Such a memory device can advantageously be included in the control unit 80 of the device 10.

1. Device (10) for the controlled release of a predefined quantity of a substance, the device (10) comprising a matrix arrangement of compartments (20) in a substrate (11), each compartment (20) being closed by at least one release mechanism (30), at least one first electrode (40) and at least one second electrode (50) being assigned to each compartment (20), the device (10) comprising a plurality of row selection lines (60) and a plurality of column selection lines (70), the number of compartments (20) exceeding the sum of the number of row selection lines (60) and the number of column selection lines (70), each first electrode (40) being electrically connected directly to one of the plurality of row selection lines (60) and each second electrode (50) being electrically connected directly to one of the plurality of column selection lines (70), wherein the release mechanism (30) has a threshold behavior.

2. Device (10) according to claim 1, wherein the release mechanism (30) is a time release mechanism (30).

3. Device (10) according to claim 1, wherein the release mechanism (30) is a closure cap.

4. Device (10) according to claim 1, wherein each compartment (20) is defined by means of one specific row selection line (61) out of the plurality of row selection lines (60) and one specific column selection line (71) out of the plurality of column selection lines (70).

5. Device (10) according to claim 1, wherein the number of compartments (20) is in the order of magnitude of the number of row selection lines (60) multiplied by the number of column selection lines (70).

6. Device (10) according to claim 5, wherein the number of rows substantially equals the number of columns.

7. Device (10) according to claim 1, wherein a first group (21) of compartments (20) is provided to contain a first quantity of a first substance and a second group (22) of compartments (20) is provided to contain a second quantity of a second substance.

8. Device (10) according to claim 4, wherein the first quantity is approximately half the second quantity.

9. Device (10) according to claim 1, wherein the release mechanism (30) of the compartment (20) is operated by means of applying an electrical potential between the first electrode (40) and the second electrode (50).

10. Device (10) according to claim 1, wherein the first electrode (40) and the second electrode (50) of each compartment (20) are electrically insulated from each other.

11. Device (10) according to claim 1, wherein the release mechanism (30) is activated by means of an electrochemical reaction.

12. Device (10) according to claim 1, wherein the release mechanism (30) is activated by means of heating the release mechanism (30).

13. Device (10) according to claim 1, wherein the device (10) comprises a control unit (80) for controlling the release of the substance.

14. Device (10) according to claim 1, wherein the number of compartments (20) is at least 100.

15. Method for controllably releasing a predefined quantity of a substance from a compartment (20) using a device (10) comprising a matrix arrangement of compartments (20) in a substrate (11), each compartment (20) being closed by at least one release mechanism (30), at least one first electrode (40) and at least one second electrode (50) being attributed to each compartment (20), the device (10) comprising a plurality of row selection lines (60) and a plurality of column selection lines (70), the number of compartments (20) exceeding the sum of the number of row selection lines (60) and the number of column selection lines (70), the method comprising the steps of:
electrically connecting the first electrode (40) of the compartment (20) directly to one of the plurality of row selection lines (60)
electrically connecting the second electrode (50) of the compartment (20) directly to one of the plurality of column selection lines (70)
applying an electrical potential above a predetermined threshold value between the first electrode (40) and the second electrode (50) by means of one of the plurality of row selection lines (60) and one of the plurality of column selection lines (70).

16. Method according to claim 15, wherein more than one compartment (20) release the substance at the same time.
